(19) United States
(12) Patent Application Publication
(10) Pub. No.: US 2025/0288211 A1
(43) Pub. Date: Sep. 18, 2025

(54) MONITORING SYSTEM COMPRISING AN ASSOCIATED MEASURING UNIT

(71) Applicant: Dräger Safety AG & Co. KGaA, Lübeck (DE)

(72) Inventors: Tilman Von Blumenthal, Lübeck (DE); Hans-Ullrich Hansmann, Lübeck (DE); Henning Gerder, Lübeck (DE); Robert Jahns, Lübeck (DE); Christoph Osterloh, Lübeck (DE)

(21) Appl. No.: 19/077,333

(22) Filed: Mar. 12, 2025

(30) Foreign Application Priority Data
Mar. 13, 2024 (DE) 102024107080.3

(51) Int. Cl. A61B 5/0205; A61B 5/00; A61B 5/024; A61B 5/1455; B64D 11/00; B64D 13/00

(52) U.S. Cl. CPC A61B 5/0205; A61B 5/14552; A61B 5/681; B64D 11/00; B64D 13/00; A61B 5/02433; A61B 2503/22

(57) ABSTRACT

A monitoring system comprising a measuring unit is described. The monitoring system may serve to monitor an aircraft pilot in an aircraft with respect to the gaseous components in the breathing gas supply and to levels of gases in the bloodstream of the aircraft pilot.

### CROSS-REFERENCE TO RELATED APPLICATIONS

This application claims the priority of German Patent Application No. 102024107080.3, filed on Mar. 13, 2024, and titled “MONITORING SYSTEM COMPRISING AN ASSOCIATED MEASURING UNIT,” which is hereby incorporated by reference in its entirety for all nonlimiting purposes.

### TECHNICAL FIELD

This disclosure relates to a monitoring system for monitoring an aviator in an aircraft. The monitoring system is used to monitor the breathing gas supply of an aviator in an aircraft.

### BACKGROUND

The term “aircraft” or “flying machines” is understood to mean airplanes or helicopters used in civil or military aviation, such as passenger airplanes in scheduled or charter flights, as well as ultra-fast airplanes traveling close to or above supersonic speeds. In particular, flights with jet planes at supersonic speeds and/or at altitudes of more than 15,000 meters above sea level place great demands on the flight crew's fitness to fly. For the purposes of this disclosure, the term “aviator” includes pilots, co-pilots, airmen, flight engineers, on-board engineers and other flight crew. An aviator must be fit-to-fly, which includes physical and mental fitness, a good level of attentiveness, the ability to concentrate and alertness at all times at high altitudes, in rapid flight maneuvers or flight attitudes such as turning, diving, inverted flying at high speeds and high accelerations. In addition to the personal and health conditions of the aviator and reliable equipment of the airplane, securely supplying the aviator with perfect and safe breathing air is also very important for a safe flight.

Paramagnetic methods are often used to determine oxygen concentration in gases. A basic principle for measuring oxygen in a measuring chamber using heat conduction changes in conjunction with paramagnetism is described in U.S. Pat. No. 6,430,987B1.

An infrared optical carbon dioxide sensor, a so-called IR carbon dioxide sensor, is known from U.S. Pat. No. 8,399,839 B.

In addition to the information as to whether and in what quantities oxygen is supplied to the aviator in a breathing gas mixture, it is of interest whether and how much of the supplied oxygen reaches the aviator's bloodstream. It is also of interest whether, in addition to levels of oxygen in the aviator's bloodstream, there are also significant levels of undesirable gases in the aviator's bloodstream.

In the context of use in an aircraft, any sensor system whose functionality can be significantly impaired by vibrations, accelerations or pressure changes is not preferred. Radio-based solutions are also not always preferred for sensor system to be used in aircraft, as ensuring that the radio waves emitted by the sensor system do not have a negative impact on the aircraft's on-board electronics can usually only be achieved with a great amount of effort.

U.S. Pat. No. 10,561,863 B1 describes a measuring system with the possibility of measuring oxygen saturation (SPO₂) in the blood. Such a measurement can be made possible, for example, by a sensor arranged on a finger, foot or ear.

An optical sensor is used to determine the oxygen saturation (SPO₂) in the blood.

A monitoring system for monitoring pilots is known from US 2021 0405008 A1, which is hereby incorporated by reference in its entirety. The monitoring system is preferably designed as part of the aviator's equipment as a stand-alone and mobile unit worn on the body with its own power supply.

The object that arises from the prior art is that of making it possible to check whether oxygen supplied to the aviator with the breathing gas enters the aviator's bloodstream.

### SUMMARY

This object is achieved by a monitoring system comprising an associated measuring unit for measuring oxygen saturation as described herein.

Further features and details of this disclosure can be found in the dependent claims, the description and the drawings.

A monitoring system according to this disclosure is designed to monitor a composition of a breathing gas mixture of a breathing gas supplied to an aviator in aircraft or flying machines and has a sensor system, a module for transporting gas, a module for checking the quantity of gas and a checking and evaluation unit.

Aspects of this disclosure are disclosed in embodiments of a monitoring system which has at least one sensor system, a module for transporting gas, a checking and evaluation unit and a measuring unit.

### BRIEF DESCRIPTION OF THE DRAWINGS

In the drawings:

FIG. 1 shows a monitoring system according to the prior art;

FIG. 2 shows a monitoring system having a measuring unit according to one or more arrangements; and

FIG. 3 shows a variant of a monitoring system having a measuring unit according to one or more arrangements.

### DETAILED DESCRIPTION

A sensor system as described herein comprises at least one gas sensor. The at least one gas sensor is designed to determine oxygen (O₂) levels. The at least one gas sensor can also be designed to determine levels of moisture or water vapor (H₂O), nitrogen (N₂), carbon dioxide (CO₂) or carbon monoxide (CO) in the breathing gas mixture. The sensor system can also have a moisture sensor for determining the moisture level in the breathing gas mixture, which, in combination with the checking and evaluation unit, is designed to determine a value that indicates the moisture level or level of water in the breathing gas mixture.

The sensor system can also have a pressure sensor for determining a pressure level in the breathing gas mixture, which, in combination with the checking and evaluation unit, is designed to determine a value that indicates the pressure level of the breathing gas mixture. The sensor system can further comprise a temperature sensor for determining a temperature of the breathing gas mixture, which, in combination with the checking and evaluation unit, is designed to determine a value that indicates a temperature of the breathing gas mixture.

The checking and evaluation unit is designed to organize, check or use open-loop or closed-loop control to control the process of monitoring the breathing gas supply. The checking and evaluation unit is further designed to check the gas transporting module and the module for checking the quantity of gas.

The gas transporting module is designed to supply defined quantities of breathing gas mixture from a measuring location to the monitoring system and to the sensor system by a sample gas line.

The monitoring system comprises a measuring unit. In combination with an optical sensor arranged on a finger, wrist or ear of the aviator, the measuring unit is designed to measure blood components, in particular blood gas components, of the aviator. The measuring unit has components for operating the optical sensor, such as a drive circuit for driving red and infrared radiation sources in the optical sensor, such as LEDs for emitting light of wavelengths of 660 nm and 920-940 nm. In addition to the LED radiation sources, the optical sensor has light detection components, such as photodiodes, phototransistors or photoresistors, for receiving the red and infrared radiation.

The measuring unit comprises signal amplification, signal processing, signal filtering and signal conversion components. The optical sensor is designed to illuminate or transilluminate blood vessels, for example in the ear or earlobe, by emitting light of different wavelengths in the wavelength range of approximately 500 nm to 1000 nm (red to infrared). The light influenced by specific absorption in the blood vessels can be detected by a transmitted light arrangement or a reflective arrangement. The specific absorption of light of different wavelengths in the blood vessels allows for an analysis of the blood gas components in the aviator's bloodstream.

The measuring unit is thus designed to cooperate with the optical sensor to determine the saturation level of at least one blood-soluble gas in the aviator's bloodstream.

The basis of the following considerations with explanations regarding determining blood oxygen saturation is a measuring method with two typical wavelengths: 660 nm and 920-940 nm. Using two wavelengths, the oxygen saturation in the blood can be determined on the basis of Formula 1. Measuring apparatuses that can perform a blood gas analysis with two wavelengths are also called pulse oximeters.

$${S}_{p} {O}_{2} = \frac{{Hb O}_{2}}{{Hb O}_{2} + Hb} \qquad Formula 1$$

with:
HbO2 for the concentration of oxygenated hemoglobin (oxyhemoglobin),
Hb for deoxygenated hemoglobin (deoxyhemoglobin).

The measuring principle involving the evaluation of two wavelengths works on the basis of the following physical conditions and relationships:
At 660 nm, the signal absorption (extinction coefficient) for hemoglobin molecules without oxygen bound thereto (deoxyhemoglobin, reduced hemoglobin) is 10 times higher than for hemoglobin molecules with oxygen bound thereto (oxyhemoglobin).
At 940 nm, the signal absorption (extinction coefficient) for hemoglobin molecules without oxygen bound thereto (deoxyhemoglobin, reduced hemoglobin) is 3 times lower than for hemoglobin molecules with oxygen bound thereto (oxyhemoglobin).

Using light of four different wavelengths, the oxygen saturation in the blood can be determined on the basis of Formula 2, even if other components are present in the blood. Measuring apparatuses that can perform a blood gas analysis with four wavelengths are also called CO-oximeters.

$${S}_{p} {O}_{2} = \frac{{Hb O}_{2}}{{Hb O}_{2} + Hb + CO Hb + MetHb} \qquad Formula 2$$

in which:
CO-Hb is the proportion of hemoglobin in the blood that is occupied by carbon monoxide (CO)
Met-Hb is methemoglobin.

Met-HB and CO-Hb are also called Dys-HB (dysfunctional hemoglobin), i.e., a hemoglobin derivative that is unable to reversibly bind to oxygen and therefore cannot transport oxygen from the lungs to the cells.

In carboxyhemoglobin (CO-Hb), carbon monoxide (CO) is covalently bound to the hemoglobin molecule. In carbaminohemoglobin (CO₂-Hb), carbon monoxide (CO₂) is bound to the hemoglobin molecule. In methemoglobin (Met-Hb), the Fe²⁺is oxidized to form Fe³⁺. The measured values from CO-oximeters and pulse oximeters may sometimes differ significantly if significant levels of carbon monoxide (CO) are present in the blood. In such a case, a pulse oximeter indicates that oxygen saturation is too high. The excessive SPO₂ reading can be caused by levels of dyshemoglobin that may be present in the blood, which cannot be distinguished from levels of oxyhemoglobin using the two wavelengths of a pulse oximeter. To determine whether there are levels of dyshemoglobin, in the form of carboxyhemoglobin (CO-Hb) or methemoglobin (Met-Hb), present in the aviator's blood, a measurement with a CO-oximeter that uses four wavelengths is necessary.

EP 2 813 180 B1 discloses solutions to differentiate between different types of hemoglobin by means of a measuring technique that uses four different wavelengths.

For monitoring the blood composition or determining blood gases in the blood of aviators during operations, it is important that the measurement for the oxygen saturation is not distorted by levels of dyshemoglobin. In addition, for monitoring the blood composition or determining blood gases in the blood of aviators during a flight, it is of great interest whether levels are present in the blood and, if so, which ones. This applies in particular to carboxyhemoglobin (CO-Hb) levels in the blood.

Carboxyhemoglobin (CO-Hb) levels indicate that the aviator has inhaled carbon monoxide. Increased concentrations of CO-HB can therefore indicate carbon monoxide intoxication (CO poisoning), which can be caused in particular by inhaling amounts of carbon monoxide found in exhaust gases or smoke gases. Exposure to CO increases the concentration of CO-Hb in the blood. Inhaled carbon monoxide has an affinity for hemoglobin that is about 300 times higher than that of oxygen. Therefore, a CO concentration of just 0.1% in the inhaled air is sufficient to occupy about 50% of the total hemoglobin with CO.

The normal range for carboxyhemoglobin (SpCO) is 0.4% to <1.6%.

Effects and symptoms of increased amounts of carboxyhemoglobin in the blood are:

| | |
|---|---|
| CO-HB < 5%: | headache |
| CO-HB between 6% to 20%: | shortness of breath, breathlessness |
| CO-HB between 21% and 30%: | nausea, dizziness, fatigue |
| CO-HB between 31% and 40%: | nausea, dizziness, confusion |
| CO-HB between 41% and 50%: | effect on cardiac function (syncope, |
| | tachycardia) |
| CO-HB between 51% and 60%: | seizures, shock, apnea, coma |
| CO-HB > 65%: | danger of death |

Inhalation of exhaust gases or smoke gases by the aviator may occur during flight, for example if the aircraft's gas supply system malfunctions, whereby the outside air drawn in for supplying breathing gas may contain certain amounts of smoke gases or exhaust gases, for example caused by aircraft flying ahead.

Increased concentrations of Met-HB can lead to methemoglobinemia with an increase in the methemoglobin level in the blood and a lack of oxygen to, for example, tissues, organs, and the brain. Methemoglobinemia can be caused, for example, by inhalation of gases or vapors of aniline or nitrobenzene, such as fumes from paints, varnishes or solvents.

The normal range of Met-HB is 1% to less than 2% of total hemoglobin.

Effects and symptoms of increased amounts of methemoglobin in the blood include:

| | |
|---|---|
| Met-HB < 15%: | no complaints |
| Met-HB between 15% and 20%: | headache, dizziness |
| Met-HB between 20% and 40%: | nausea, cyanosis |
| Met-HB > 45%: | seizures, confusion |
| Met-HB > 70%: | danger of death |

Inhalation of solvent vapors by the aviator may occur during a flight, for example, in the event of malfunctions on or in the pipeline system of the aircraft's gas supply system, in which case quantities of solvents, operating materials, auxiliary materials or lubricants may then enter the airflow used in the breathing gas supply.

Embodiments show at which positions the optical sensor can be arranged on the aviator's body. Possible positions for the optical sensor include positions on the ear, for example the earlobe, on the finger, the wrist and, in principle, also the foot or ankle. The use of a position on the ear or earlobe is preferred, especially if the monitoring unit is positioned in a vest worn on the aviator's body or the measuring unit is positioned on the aviator's oxygen mask, helmet or headphones, since in this case only short distances have to be bridged between the optical sensor and the measuring unit as well as between the monitoring unit and the measuring unit.

The ear sensor should therefore be arranged on the pilot's ear in or as close as possible to the pilot's face mask, communication equipment (headphones) or helmet.

In certain embodiments, ear sensors can also be arranged on both of the pilot's ears, for example to carry out a simple measurement on one ear that is based on 2 wavelengths to determine % SpO₂ and to determine % SPCO, % SPMet on the other ear by means of a sensor with more than 4, preferably at least 7, wavelengths.

In this way, the above-mentioned differences between functional saturation measured by pulse oximetry and fractional saturation measured by the CO-oximeter can be identified in order to obtain a direct indication of dyshemoglobinemia should a difference exist. In addition, by using sensors on both ears, temporary artifacts that occur at only one ear sensor can, on average, be reduced over time. This advantage also applies to the use of a CO-oximeter on each ear.

The optical sensor can be attached to the wrist, for example, in the form of a watch or heart rate monitor watch. The watch or heart rate monitor watch can comprise the optical sensor and the measuring unit. The watch or heart rate monitor watch can function as a kind of pulse oximeter or a CO-oximeter and can also be used to output measured values, information or alarms from the pulse oximeter or the CO-oximeter to the aviator in the form of acoustic or visual alarms or as a vibration alarm.

In some embodiments, the measuring unit can cooperate with the optical sensor to form a pulse oximeter or a CO-oximeter.

The pulse oximeter functions as measurement technology with two different wavelengths as previously described on the basis of Formula 1.

The CO-oximeter functions as measurement technology with four different wavelengths as previously described on the basis of Formula 2.

Pulse oximetry can be used to measure, determine and/or monitor oxygen O₂ saturation levels in the aviator's bloodstream.

CO-oximetry can be used to measure, determine and/or monitor levels of different types of hemoglobin (HB) in the aviator's bloodstream. These include, for example and in particular, O₂-HB (oxyhemoglobin), Dys-HB such as Met-HB (methemoglobin), CO-HB (carboxyhemoglobin) or S-HB (sulfhemoglobin).

In certain embodiments, the measuring unit is designed to cooperate with the optical sensor and determine an oxygen saturation level (SPO₂) and/or a saturation level of at least one other gas that is soluble in the blood, in particular a saturation level (SPCO) of carbon monoxide or a saturation level (SPCO₂) of carbon dioxide in the aviator's bloodstream.

In certain embodiments, in order to determine the saturation level in the aviator's bloodstream, the measuring unit can be designed to cooperate with the optical sensor to analyze the level of at least one type of hemoglobin in the bloodstream.

In certain embodiments, the at least one gas sensor can be used to determine the level of carbon dioxide CO₂ and/or carbon monoxide CO in the breathing gas mixture.

In certain embodiments, levels of different types of hemoglobin in the aviator's bloodstream in conjunction with information about the aviator's cardiovascular system can be determined and/or monitored.

For this purpose, the measuring unit can be designed to cooperate with the optical sensor
to determine a heart rate
and/or to determine heart rate variability.
and/or to determine blood flow in the blood vessels of the aviator.

The heart rate allows conclusions to be drawn about the physical strain on the aviator. This allows the checking and evaluation unit to take the heart rate into account when determining the aviator's condition. Heart rate variability allows conclusions to be drawn about the psychological strain on the aviator. This allows the checking and evaluation unit to take heart rate variability into account when determining the aviator's condition. The current blood flow in the blood vessels can be evaluated by the checking and evaluation unit in addition to oxygen saturation as an indicator of a state of shock of the cardiovascular system.

In certain embodiments, the checking and evaluation unit can be designed as a plurality of checking modules. A checking module can be designed as a component of the monitoring system. A checking module can be designed as a component of the measuring unit. In such embodiments, the measuring unit can be arranged in or on the monitoring system as a checking module having an electronic unit, or can be assigned to the monitoring system. The electronic unit can be arranged on the aviator's equipment, such as the mask, helmet or headphones, with an advantageous short cable connection to the optical sensor on the aviator's ear. The measuring unit and/or the electronics unit can be connected to the monitoring system-which is preferably arranged in a vest on the aviator-by means of a further short cable connection. Alternatively, the measuring unit and/or the electronic unit can be connected to the aviator's monitoring system via a wireless connection, such as Bluetooth.

In certain embodiments, components of the monitoring system and measuring unit can be connected to one another by means of at least one interface, and the checking and evaluation unit and/or the checking modules can be designed to coordinate the operation of the measuring unit and of the monitoring system by means of the interfaces. For this purpose, at least one interface can be arranged in or on the monitoring system and/or the measuring unit. The interface is preferably designed as a power and/or data interface. The checking and evaluation unit can be designed to coordinate the measuring unit and the monitoring system by means of the interfaces. In these embodiments, components of the monitoring system and measuring unit can be connected to one another by means of interfaces for transferring power and/or data. Data can be transferred unidirectionally from the monitoring system to the measuring unit as well as bidirectionally between the monitoring system and the measuring unit. By means of a data connection from the monitoring system to the measuring unit, the monitoring system can, for example, drive or trigger the measuring unit so as to activate a measurement. By means of a data connection from the measuring unit to the monitoring system, the measuring unit can, for example, drive or trigger the activation of actions such as measurements or alarms. The interface between the monitoring system and the measuring unit can be wired or wireless. The interface between the measuring unit and the optical sensor can be wired or wireless.

In certain embodiments, the checking and evaluation unit and/or one of the checking modules can be designed to compare at least two elements of the group and/or to compare at least one element of the group with at least one comparison value:
saturation level (SPO₂) of oxygen in the aviator's bloodstream;
saturation level (SPCO) of carbon monoxide in the aviator's bloodstream;
saturation level of at least one other blood-soluble gas in the aviator's bloodstream;
level of at least one type of hemoglobin in the aviator's bloodstream;
level of oxygen O₂ in the breathing gas mixture;
level of carbon dioxide CO₂ in the breathing gas mixture;
level of carbon monoxide CO in the breathing gas mixture;
aviator's heart rate;
aviator's heart rate variability; and
blood flow in the aviator's blood vessels;
   and to determine the aviator's condition on the basis of the comparison.

In certain embodiments, the checking and evaluation unit and/or one of the checking modules can be designed to determine the state of a breathing gas supply of the aircraft on the basis of the comparison.

In certain embodiments, an output unit can be arranged in or on the monitoring system or assigned to the monitoring system, which allows the provision of at least one output signal which indicates the aviator's condition and/or indicates the state of the breathing gas supply of the aircraft and/or indicates the state of the measuring unit of the optical sensor or the monitoring system.

States of the measuring unit, the optical sensor and the monitoring system can each indicate operational readiness or malfunctions. States of the breathing gas supply can each indicate operational readiness, disturbances or malfunctions.

Conditions of the aviator may indicate the state of the aviator's health. Health assessments can be made on the basis of a determined composition of hemoglobin and/or hemoglobin derivatives (oxyhemoglobin, deoxyhemoglobin, dyshemoglobin) or on the levels or saturation levels of oxygen, carbon monoxide, carbon dioxide and/or other components in the aviator's bloodstream. Health assessments can be made on the basis of a gas composition that is determined in the aviator's breathing gas supply.

Health assessments can be made on the basis of the determined composition of hemoglobin and/or hemoglobin derivatives, levels or saturation levels in the aviator's bloodstream and the gas composition determined in the aviator's breathing gas supply.

In certain embodiments, an external input/output unit can be assigned to the monitoring system. Preferably, the monitoring system and the external input/output unit are connected to one another by means of a wireless data connection. The external input/output unit can be designed to signal events of the measurement-based monitoring process and/or results of the concentration of oxygen in the breathing gas measured by the sensor system and/or results of the composition of hemoglobin and/or hemoglobin derivatives (oxyhemoglobin, deoxyhemoglobin, dyshemoglobin) measured by the optical sensor cooperating with the measuring unit and the sensor or of levels or saturation levels of oxygen, carbon monoxide, carbon dioxide and/or other components in the aviator's bloodstream.

In such an embodiment, the external input/output unit can be designed as a heart rate monitor watch or as a wristwatch worn on an aviator, pilot or co-pilot's body, in particular that is worn on the wrist. The watch or heart rate monitor watch as an external input/output unit can allow wireless unidirectional data exchange from the monitoring system to the watch-for example for issuing an alarm-as well as bidirectional wireless data exchange between the watch and the monitoring system, which allows remote operation or open-loop remote control of the monitoring system through inputs on the watch.

In certain embodiments, the external output unit can be designed on the head of an aviator, pilot or co-pilot, in particular headphones arranged on the ear or as a structure-borne sound receiver. Headphones or structure-borne sound receivers as external input/output units can allow wireless unidirectional data exchange from the monitoring system to headphones or structure-borne sound receivers, for example for issuing an alarm.

Wireless data exchange can be implemented both bidirectionally and unidirectionally using a Bluetooth data connection in accordance with various designs according to the standard IEEE 802.15. This is made possible by various application-specific profiles within the standard IEEE 802.15.

By means of a Bluetooth data connection, a unidirectional or bidirectional data connection between the monitoring system of the pilot or copilot and the external output unit or input/output unit of the pilot or copilot can be allowed as a 1:1 connection (point-to-point) as well as a data network (mesh) between a plurality of participants, for example between the two monitoring systems of the copilot and pilot with the two input/output units or output units of the copilot and pilot. This makes it possible, for example, for a pilot's monitoring system to send signals or alarms to the co-pilot's monitoring system and/or for the co-pilot's monitoring system to send signals or alarms to the pilot's monitoring system. For example, the wireless data connection can also be made possible communication between the pilot or co-pilot's monitoring system and at least one other participant in a data network. A further participant in the data network can, for example, be a flight engineer in the same aircraft or be designed as an interface module for connection to the on- board electronics or connection to an aircraft radio apparatus.

In certain embodiments, the measuring unit can be arranged in one structural unit together with the optical sensor as a component of a heart rate monitor watch on the aviator's wrist. In such an embodiment, the heart rate monitor watch can advantageously be connected to the monitoring system for data exchange, for example by means of a wireless connection or radio connection.

In certain embodiments, the external input/output unit can be designed to carry out at least one action from the following group of actions when the measuring unit, monitoring system and optical sensor interact:
activation and/or deactivation of the measuring unit;
activation and/or deactivation of the monitoring system;
activation and/or deactivation of wireless communication between the measuring unit and the monitoring system;
activation and/or deactivation of wireless communication between the measuring unit and the optical sensor;
activation and/or deactivation of wireless communication between the monitoring system and the optical sensor.

In certain embodiments, an additional sensor system can be arranged in the monitoring system which is designed to determine and provide events and/or situations during the flight. The additional sensor system can, for example, comprise an acceleration sensor, for example in the form of a 2-or 3-axis acceleration sensor (accelerometer), a compass sensor (electronic compass, gyrocompass, fluxgate compass), an altitude sensor (altimeter) or a gyro sensor (gyrometer). By means of the additional sensor system, flight maneuvers and flight situations (take-off, landing, descent, acceleration, looping, refueling in the air) can be identified.

In the following, a brief summary is given of some of the advantages of this disclosure which result from the interaction of monitoring an aviator using breathing gas analysis and blood gas analysis by means of a CO-oximeter with regard to monitoring pilots/co-pilots during a flight. By detecting Met-HB, for example, if an increase in Met-HB is detected, Met-HB threshold values can be used to identify whether there may be solvent fumes from plastics material parts (plasticizers) in the breathing gas supply during the flight. By combining and comparing data with data from the acceleration sensor in the monitoring system, the correspondingly associated points in time and/or events and/or flight maneuvers during the flight can then be identified and marked.

Combining the detection of gases (O₂, CO₂, CO, etc.) in the supply (mask) of air to the aviator for inhaling as well as in the cockpit and their further analysis with additionally recording concentrations or saturation levels of gases (O₂, CO) and any other substances in the blood using a CO-oximeter module on the ear or ears of the pilot/co-pilot, if necessary, has a number of other advantages. The checking and evaluation unit can thus make a comparison between O₂ breathing gas concentrations and O₂-HB, a comparison between CO₂ breathing gas concentrations and CO₂HB and/or a comparison between CO breathing gas concentrations and CO-HB.

By recording CO₂-HB and/or CO-HB together with correspondingly measuring CO₂ and/or CO in the breathing gas supply by means of the monitoring system, if an increase in CO₂-HB or CO-HB is detected by a comparison and optionally by taking into account threshold values, it is, for example, possible to identify whether exhaust gas components may have entered the pilot's breathing gas supply during the flight.

By recording O₂-HB and correspondingly measuring O₂ in the breathing gas supply, if a drop in O₂-HB at a constant O₂ concentration in the breathing gas supply is detected by a comparison and optionally by taking into account threshold values, it is, for example, possible to identify whether the pilot or co-pilot may not have positioned the oxygen mask correctly or with an inadequate seal on the face.

Data from additional sensor systems, such as an acceleration sensor, can be used to classify CO-oximeter measured values, e.g., as “valid” or “invalid.”

The data from the acceleration sensor or gyrometer can allow the checking and evaluation unit to mark certain situations, possibly implausible, inaccurate or invalid measured values from the CO-oximeter module, during the flight of the checking and evaluation unit and thus reduce measurement artifacts with a similar, specific flight-related effect. The use of an acceleration sensor or gyrometer in the monitoring unit does not allow for signal improvement of the measured data from the CO-oximeter per se. Influences, for example due to a flight maneuver (e.g., looping), are not eliminated from the measured signal. However, the checking and evaluation unit can perform “temporal blanking” or “fading out” for points in time or time intervals identified as being concise by the marking made on the basis of the acceleration sensor or gyrometer.

This disclosure will be explained in more detail using the following description with reference being partially made to the drawings.

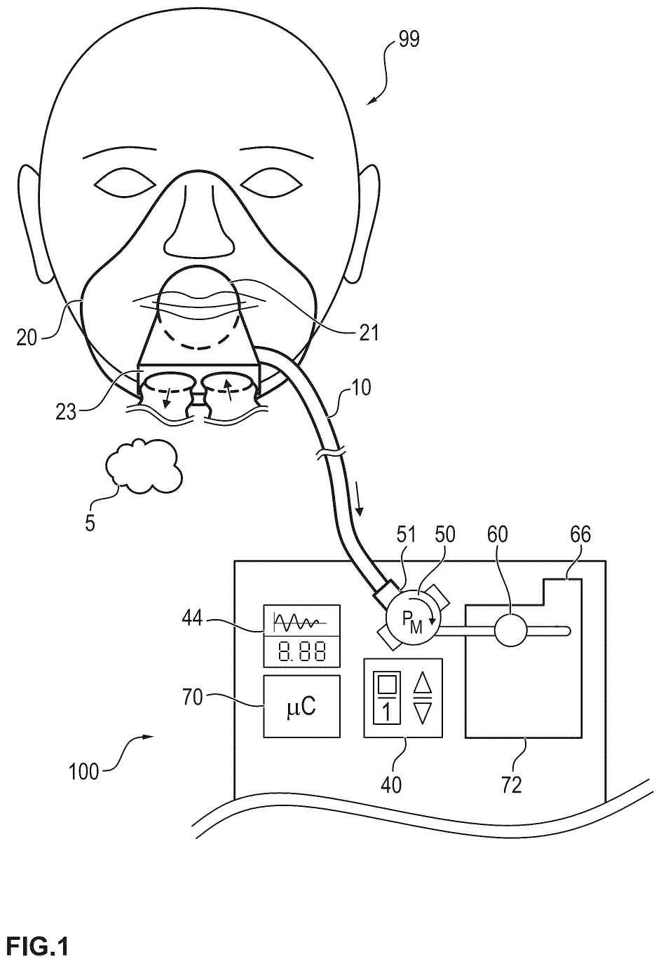

FIG. 1 is a schematic view of a monitoring system 100 according to the prior art based on US20210405008 A1.

The monitoring system 100 is connected by a measuring gas line 10 to an aviator (pilot, co-pilot) 99 by means of an oxygen mask 20. The breathing mask 20 has a gas connection 21, a connection element 23 and hose lines 24, 25. The tube lines 24, 25 are used to discharge and supply breathing gases to and from the aviator 99. The breathing gases can be discharged into a system of the aircraft provided and intended for this purpose or into the environment 5, such as the cockpit of the aircraft. The monitoring system 100 comprises operating elements 40, display elements 44, a gas delivery module 50 and a measuring device 66.

The measuring device 66 has a sensor system 60, in particular a gas analyzing sensor system 60, but can also have further sensor systems 60, for example a sensor system 60 which is designed to measure temperatures, pressures or moisture.

The gas delivery module 50 is preferably designed as a pump PM.

In addition, the monitoring system 100 has a checking and evaluation unit 70.

The operating elements 40, the display elements 44, the sensor system 60, the gas delivery module 50 are connected to the checking and evaluation unit 70 and, if necessary, to one another via signal and data lines or checking lines (not shown in said FIG. 1). These checking lines or signal and data lines can, for example, be designed as a bus system (CAN) or network.

The checking and evaluation unit 70 is designed and provided to check and/or drive the gas delivery module 50 in such a way that breathing gases are delivered from the oxygen mask 20 to the measuring device 66 through the sample gas line 10 and a gas inlet 51.

Thus, a quantity or partial quantity of breathing gas 10 is then available to the measuring device 66 in order to analyze it using measurements and to provide it to the checking and evaluation unit 70 as measured values.

The checking and evaluation unit 70 makes it possible to evaluate and process the measured values and to display them on display elements 44.

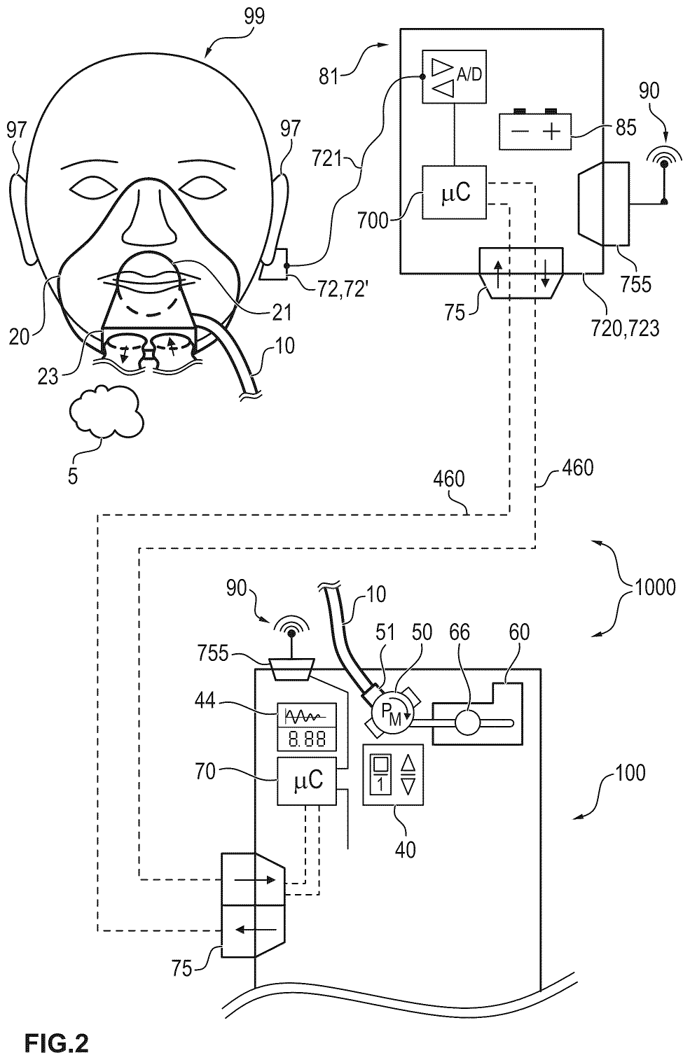

FIG. 2 schematically shows a measuring unit 81 in an arrangement 1000

having a monitoring system 100 according to FIG. 1. Identical elements in FIGS. 1 and 2 are designated by identical reference signs in FIG. 1 and FIG. 2. An optical sensor 72 is shown as an ear sensor attached to an ear 97 of a pilot 99. The optical sensor 72 is connected to the measuring unit 81 by a connecting cable 721. The measuring unit 81 has a checking unit 700 and a power supply 85.

The optical sensor 72 is based on the use of two optical wavelengths and is designed in a minimum configuration having a checking unit 700 to form a pulse oximeter module in order to measure the heart rate (HR, pulse) and an oxygen saturation SPO₂ or the proportion of oxyhemoglobin (O₂-HB) of the total hemoglobin (oxyhemoglobin+deoxyhemoglobin+dyshemoglobin) of the pilot 99 and provide it at one of the interfaces 75, 755.

The optical sensor in a variant 72 extended to four optical wavelengths and the checking unit 700 can, in an extended configuration, be designed to form a CO-oximeter module 723 and, in addition to heart rate (HR, pulse), oxyhemoglobin and deoxyhemoglobin, can measure further hemoglobin derivatives (Dys-HB, such as CO-HB, Met-HB, S-HB, O₂-HB, CO₂-HB) of the pilot 99 and provide them at one of the interfaces 75, 755.

The measuring unit 81 or the pulse oximeter module 720 or the CO-oximeter module 723 are connected to the system 100 for monitoring pilots 99 by data lines 460 and interfaces 75 or by wireless interfaces 755 with corresponding transmitting/receiving components 90.

In optional embodiments, the pulse oximeter module 720 or the CO-oximeter module 723 can be connected directly to the oxygen mask 20 as one module.

Alternatively, the pulse oximeter module 720 or the CO-oximeter module 723 can also be designed as an element or part of the monitoring system 100.

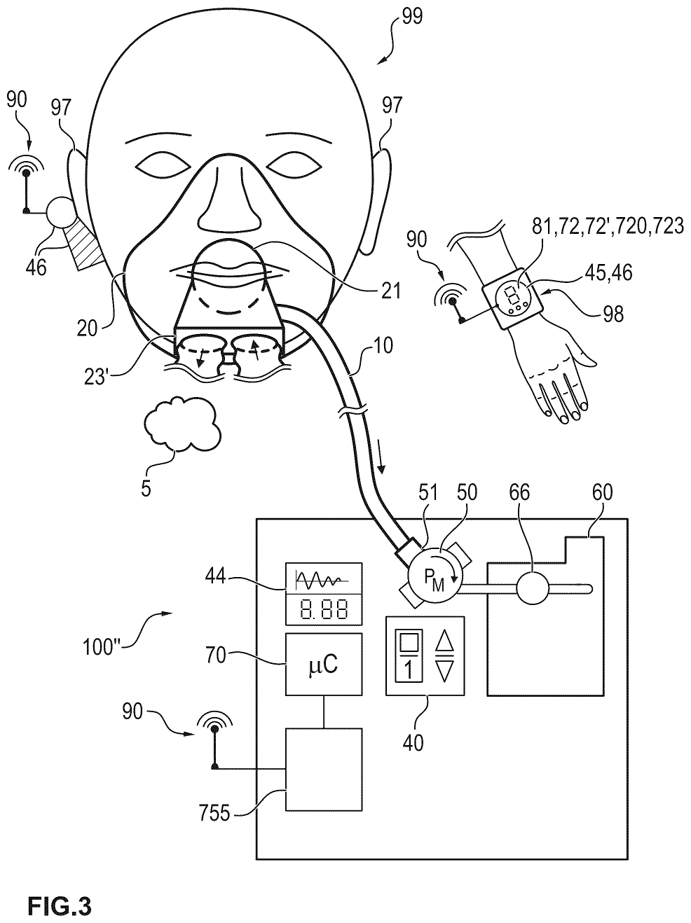

FIG. 3 schematically shows a measuring unit 81 arranged as a bracelet on the wrist 98 of a pilot 99 or co-pilot and designed as a watch 45 or wristwatch, which can have an optical sensor 72, 72′, 720, 723 and functions 700 for recording measured measuring values, such as functions for providing, outputting or forwarding information or measured values, a wireless interface 755 with corresponding transmitting/receiving components 90, data output functions 44 and/or data input functions 40 in one structural unit. Identical elements in FIGS. 1, 2 and 3 are designated by identical reference signs in FIGS. 1, 2 and 3.

In an optional extension of the monitoring system 100 or the measuring unit 81, an external output unit 46 with corresponding transmitting/receiving components 90 can be provided as an external wireless output unit 46. Such an external output unit can be formed, for example, by a headphone arranged on the ear 97 of the pilot 99 or co-pilot or as a structure-borne sound receiver. Such an external output unit can, for example, also be formed as an external input/output unit 46 by the watch 45 on the wrist 98 of the pilot 99 or co-pilot.

### LIST OF REFERENCE SIGNS

5 environment, cockpit
10 sample gas line
20 oxygen mask
21 breathing gas connection, gas connection to
24, 25 tube lines
40 operating elements, input elements
44 display elements, output elements
454 watch, wristwatch, heart rate monitor watch
46 external input/output unit
460 data lines
50 gas delivery module, pump PM
60 sensor system
70 checking and evaluation unit
700 checking unit
72, 72′ optical sensor, oximetry sensor, ear sensor, sensor on the ear
720 pulse oximeter module
721 connecting cable
723 CO-oximeter module
75, 755 interfaces, wired, wireless
85 power supply
90 transmission/receiving components
97 aviator's ear
98 aviator's wrist
99 pilot, co-pilot, aviator, airmen, flight crew
100 monitoring system
1000 arrangement consisting of the measuring unit and monitoring system

1-15. (canceled)

16. A monitoring system for monitoring an individual in an aircraft comprising:
at least one sensor system;
a measuring unit;
a module for transporting gas; and
at least one checking and evaluation unit,
wherein the checking and evaluation unit is configured to check:
the at least one sensor system; and
the module for transporting gas;
wherein the at least one sensor system comprises at least one gas sensor;
wherein the at least one sensor system and the checking and evaluation unit are configured to allow the measurement of gases or gas mixtures;
wherein the at least one checking and evaluation unit is configured to control a process of using measurements to monitor the gas composition of at least one of:
air in an aircraft,
breathing air in an aircraft, or
breathing gases in an aircraft;
wherein the controlling the process comprises performing at least one of:
organizing an open-loop or closed-loop control method,
checking an open-loop or closed-loop control method, or
using an open-loop or closed-loop control method;
wherein the module for transporting gas comprises a sample gas line and is configured to supply defined quantities of a breathing gas mixture from a measuring location to the monitoring system and to the at least one sensor system;
wherein the measuring location is arranged in a breathing gas supply to the individual such that the defined quantities of breathing gas mixture supplied to the monitoring system are representative of the composition of gases in the breathing gas mixture;
wherein the at least one gas sensor is configured to determine an oxygen level in the breathing gas mixture; and
wherein the measuring unit is designed such that it cooperates with at least one optical sensor to determine a saturation level of at least one blood-soluble gas in the bloodstream of the individual.

17. The monitoring system of claim 16, wherein the optical sensor is designed to be attached to at least one of:
an ear of the individual,
a finger of the individual,
or a wrist of the individual.

18. The monitoring system of claim 16, wherein the measuring unit is configured to cooperate with the optical sensor to determine at least one of:
a saturation level of oxygen;
a saturation level of carbon monoxide; or
a saturation level of carbon dioxide.

19. The monitoring system of claim 16, wherein the measuring unit is configured to cooperate with the optical sensor in order to:
determine the saturation level in the bloodstream of the individual; and analyze a level of at least one type of hemoglobin in the bloodstream.

20. The monitoring system of claim 16, wherein the at least one gas sensor is designed to determine at least one of:
a level of carbon dioxide in the breathing gas mixture, or
a level of carbon monoxide in the breathing gas mixture.

21. The monitoring system of claim 16, wherein the measuring unit is configured to cooperate with the optical sensor to perform at least one of:
determining a heart rate;
determining a heart rate variability; or
determining a rate of blood flow in the blood vessels of the individual.

22. The monitoring system of claim 16, wherein the at least one checking and evaluation unit comprises a plurality of checking modules, wherein at least one of the plurality of checking modules is configured as a component of the monitoring system or as a component of the measuring unit.

23. The monitoring system of claim 22, wherein the monitoring system further comprises at least one interface coupled to the measuring unit and wherein the at least one interface is configured to coordinate the operation of the measuring unit with at least one of:
the at least one checking and evaluation unit; and
the plurality of checking modules.

24. The monitoring system of claim 22:
wherein at least one of the at least one checking and evaluation unit or the plurality of checking modules is configured to:
compare at least one of:
at least two elements of a group of elements; or
at least one element of the group and at least one comparison value,
wherein the at least one comparison value comprises:
a saturation level of oxygen in the bloodstream of the individual;
a saturation level of carbon monoxide in the bloodstream of the individual;
a level of at least one type of hemoglobin in the bloodstream of the individual;
a level of oxygen in the breathing gas mixture;
a level of carbon dioxide in the breathing gas mixture;
a level of carbon monoxide in the breathing gas mixture;
a heart rate of the individual;
a heart rate variability of the individual; or
a blood flow in one or more blood vessels of the individual; and
determine a condition of the individual on the basis of the comparison.

25. The monitoring system according to claim 24, wherein at least one of the at least one checking, the evaluation unit, or a checking module of the plurality of checking modules is configured to determine the state of a breathing gas supply of the aircraft on the basis of the comparison.

26. The monitoring system of claim 24, wherein an output unit is associated with the monitoring system, wherein the output unit provides at least one output signal which indicates:
the condition of the individual;
the state of the breathing gas supply of the aircraft; or
a state of the measuring unit, the optical sensor, or the monitoring system.

27. The monitoring system of claim 26, wherein the output unit is configured as an external unit and is assigned to the monitoring system.

28. The monitoring system of claim 27, wherein the measuring unit is arranged in one module together with the optical sensor as a component of a heart rate monitor watch configured to be worn on the wrist of the individual.

29. The monitoring system of claim 27, wherein the external unit is configured to perform, when the measuring unit, monitoring system, and optical sensor interact at least one of:
activation/deactivation of the measuring unit;
activation/deactivation of the monitoring system;
activation/deactivation of wireless communication between the measuring unit and the monitoring system;
activation/deactivation of wireless communication between the measuring unit and the optical sensor; and
activation/deactivation of wireless communication between the monitoring system and the optical sensor.

30. The monitoring system of claim 16, wherein an additional sensor system comprising at least one of:
a 2-axis acceleration sensor,
a 3-axis acceleration sensor,
a compass sensor,
an altitude sensor, or
a gyro sensor,
is arranged in the monitoring system and is configured to determine and provide event information during a flight.

31. A method for monitoring an individual in an aircraft:
wherein the method is performed using a monitoring system comprising:
at least one sensor system;
a measuring unit;
a module for transporting gas;
at least one checking and evaluation unit,
wherein the checking and evaluation unit is configured to check:
the at least one sensor system; and
the module for transporting gas;
wherein the at least one sensor system comprises at least one gas sensor;
wherein the at least one sensor system and the checking and evaluation unit are configured to allow the measurement of gases or gas mixtures;
the method comprising:
with the at least one checking and evaluation unit, controlling a process of using measurements to monitor the gas composition of at least one of:
air in an aircraft,
breathing air in an aircraft, or
breathing gases in an aircraft;
wherein the controlling the process comprises performing at least one of:
organizing an open-loop or closed-loop control method,
checking an open-loop or closed-loop control method, or
using an open-loop or closed-loop control method;
wherein the module for transporting gas comprises a sample gas line and is configured to supply defined quantities of a breathing gas mixture from a measuring location to the monitoring system and to the at least one sensor system;
wherein the measuring location is arranged in a breathing gas supply to the individual such that the defined quantities of breathing gas mixture supplied to the monitoring system are representative of the composition of gases in the breathing gas mixture;
determining, with the at least one gas sensor, an oxygen level in the breathing gas mixture; and
determining, with the measuring unit and at least one optical sensor, a saturation level of at least one blood-soluble gas in the bloodstream of the individual.

32. The method of claim 31, wherein the optical sensor is designed to be attached to at least one of:
an ear of the individual,
a finger of the individual,
or a wrist of the individual.

33. The method of claim 31, further comprising using the measuring unit and the optical sensor to determine at least one of:
a saturation level of oxygen;
a saturation level of carbon monoxide; or
a saturation level of carbon dioxide.

34. The method of claim 31, further comprising using the measuring unit with the optical sensor in order to:
determine the saturation level in the bloodstream of the individual; and analyze a
level of at least one type of hemoglobin in the bloodstream.

35. The method of claim 31, further comprising using the at least one gas sensor to determine at least one of:
a level of carbon dioxide in the breathing gas mixture, or
a level of carbon monoxide in the breathing gas mixture.
